(19) United States
(12) Patent Application Publication
(10) Pub. No.: US 2016/0345832 A1
(43) Pub. Date: Dec. 1, 2016

(54) SYSTEM AND METHOD FOR MONITORING BIOLOGICAL STATUS THROUGH CONTACTLESS SENSING

(71) Applicant: Wearless Tech Inc, San Francisco, CA (US)

(72) Inventors: Pavan Kumar Pavagada Nagaraja, La Jolla, CA (US); Sivakumar Nattamai, San Francisco, CA (US); Rubi Vanessa Sanchez Castro, San Francisco, CA (US)

(21) Appl. No.: 15/163,611

(22) Filed: May 24, 2016

Related U.S. Application Data
(60) Provisional application No. 62/166,076, filed on May 25, 2015.

(51) Int. Cl. A61B 5/00; A61B 5/08; A61B 5/024; A61B 5/0205; A61B 5/11

(52) U.S. Cl. CPC A61B 5/0022; A61B 5/6887; A61B 5/02055; A61B 5/0205; A61B 5/1128; A61B 5/024; A61B 5/08; A61B 5/0077; A61B 5/4812; A61B 5/746; A61B 2503/04

(57) ABSTRACT

A system and method for monitoring biological status through contactless sensing that includes a sensing device with at least one video imaging device; a sensor data processing unit, wherein the sensor processing unit when in a respiratory sensing mode is configured to extract a set of primary components of motion from image data from the video imaging device; a biological signal processor that when in a respiratory sensing mode is configured to identify at least one dominant component of motion and generate a respiratory signal; and a monitor system.

### CROSS-REFERENCE TO RELATED APPLICATIONS

This Application claims the benefit of U.S. Provisional Application No. 62/166,076, filed on 25 May 2015, which is incorporated in its entireties by this reference.

### TECHNICAL FIELD

This invention relates generally to the field of biological sensing, and more specifically to a new and useful system and method for monitoring biological status through contactless multi-sensing.

### BACKGROUND

Conventional monitors have primarily been used to detect vital signs through the attachment of hardware to the subject. A patient in the hospital can often have a number of different sensors attached to various parts of the body for constant monitoring. This can be cumbersome and inconvenient. In other situations, sensing is performed periodically due to the inconvenience and discomfort caused by such contact-based sensing devices. Such contact-based sensing or periodic sensing can be undesirable in many situations. This is particularly in the case of monitoring a baby. Contact-based sensing can result in skin irritation, skin damage (e.g., in premature babies), burns, and/or potential harmful effects as a result of the close proximity of electronics. Additionally, contact-based sensing can result in numerous false alarms because of sensor leads falling off. Video and audio monitoring are generally used in a baby monitor. As a result, parents have to perform continuous checks of their baby and/or monitor at regular intervals despite having a monitor. Such un-informative baby monitoring tools can increase anxiety for a parent. Furthermore, without constant monitoring, the baby monitor may not identify health risks. Thus, there is a need in the biological sensing field to create a new and useful system and method for monitoring biological status through contactless multi-sensing. This invention provides such a new and useful system and method.

### BRIEF DESCRIPTION OF THE FIGS.

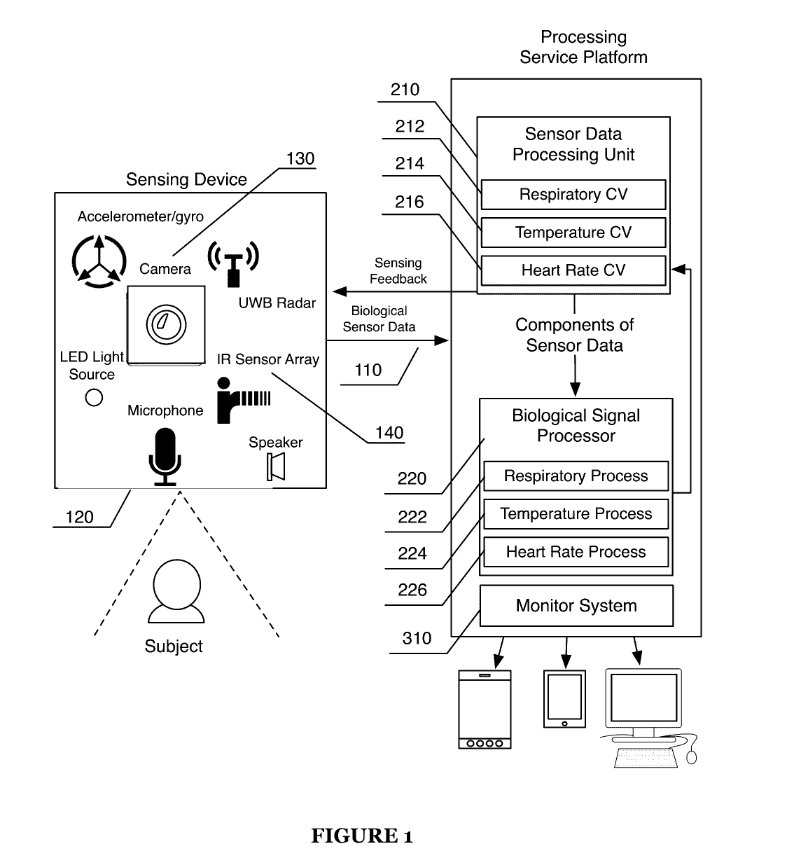

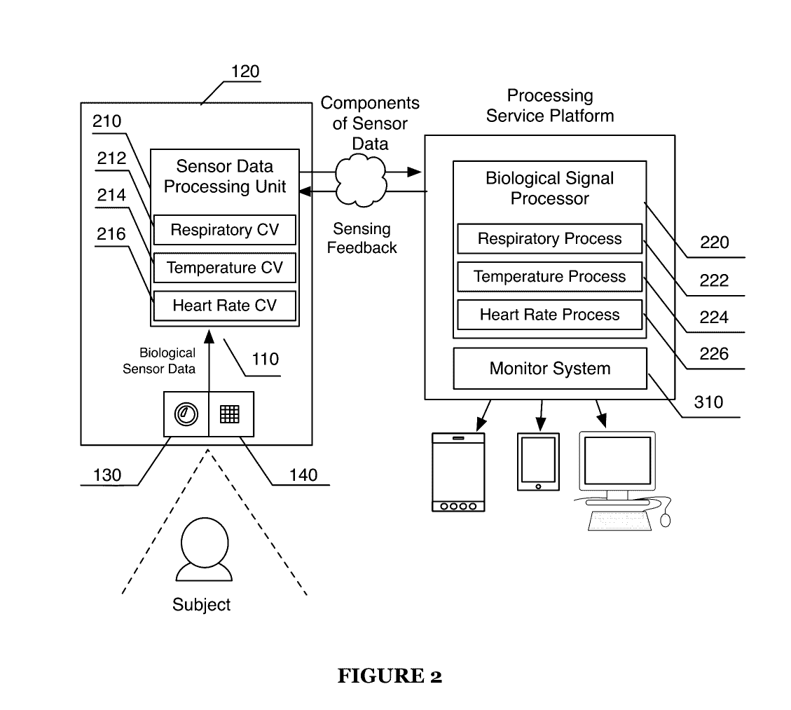

FIGS. 1 and 2 are schematic representations of systems of preferred embodiments;

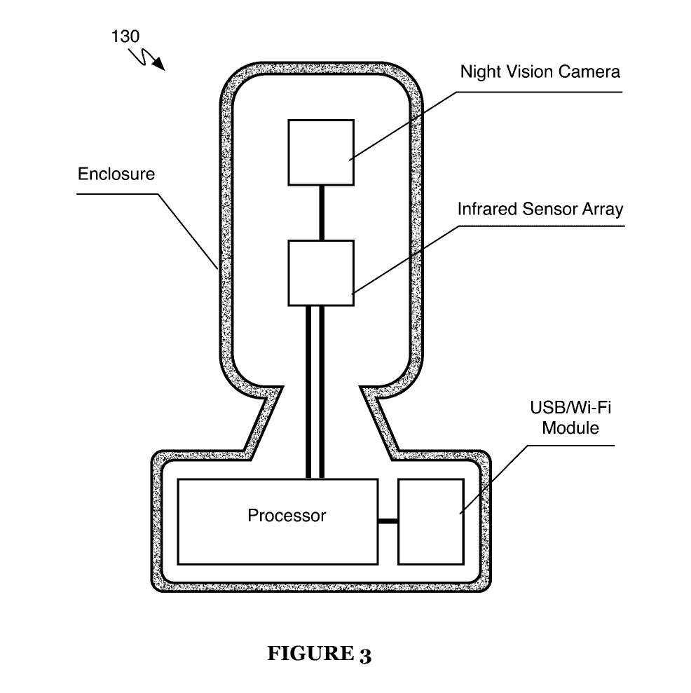

FIG. 3 is a cross-sectional schematic representation of one exemplary implementation of a sensing device;

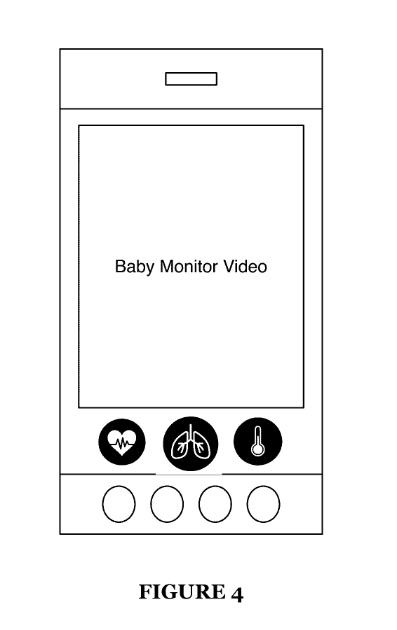

FIG. 4 is a schematic representation of an exemplary monitoring system;

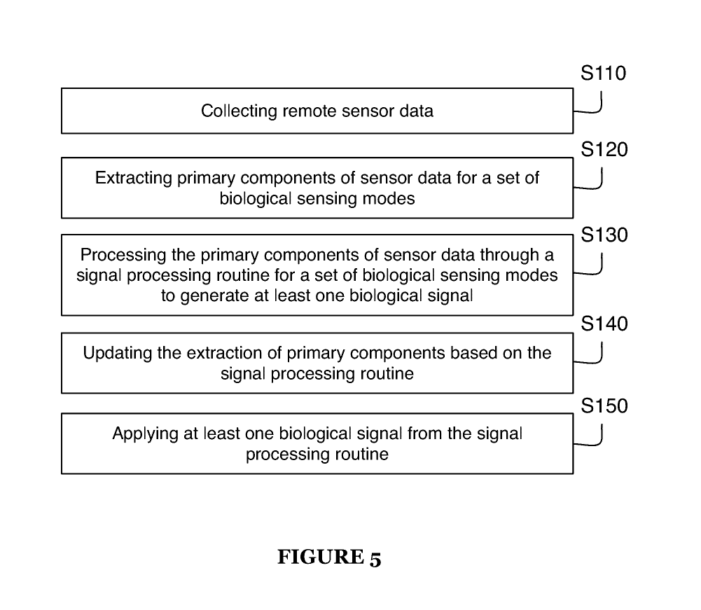

FIG. 5 is a flowchart representation of a method of a preferred embodiment;

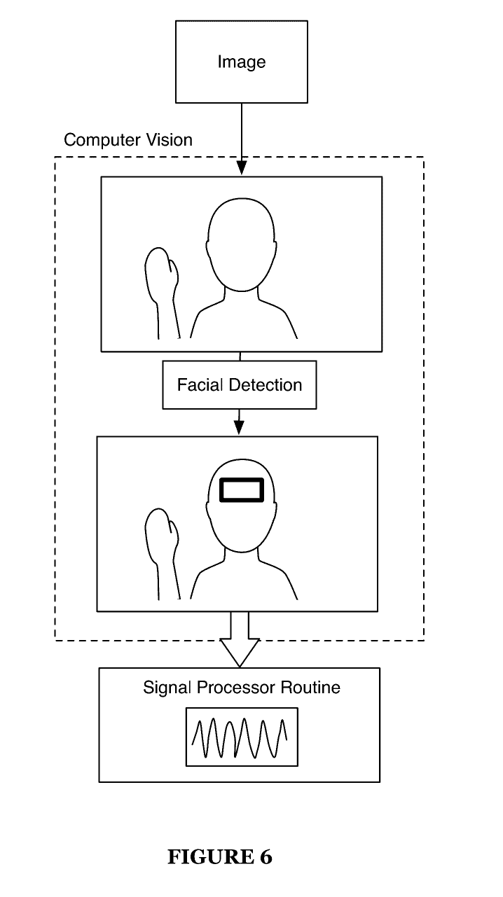

FIG. 6 is a schematic representation of utilizing facial detection;

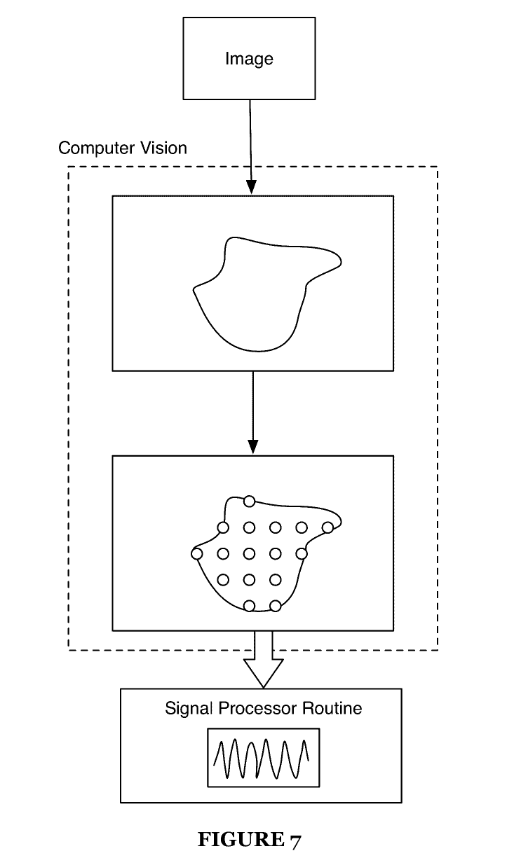

FIG. 7 is a schematic representation of extracting and processing a set of primary components of sensor data;

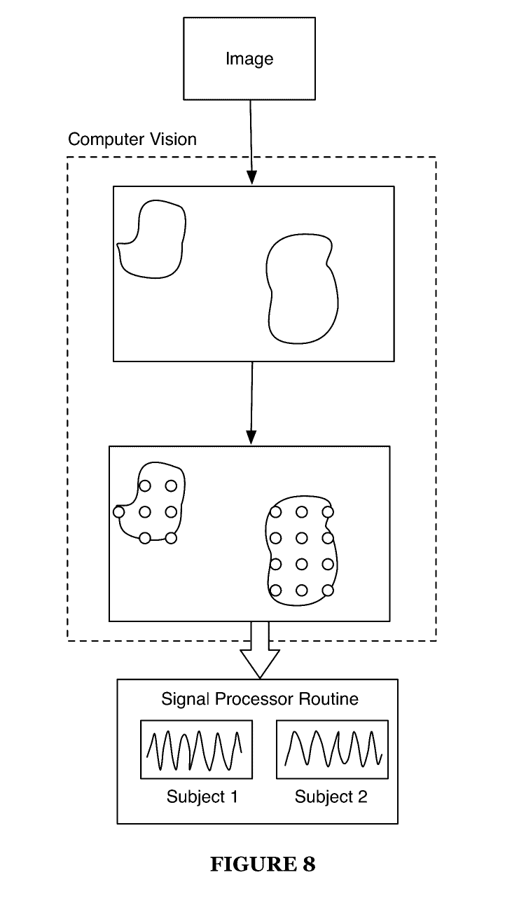

FIG. 8 is a schematic representation of extracting and processing a set of primary components of sensor data for multiple subjects;

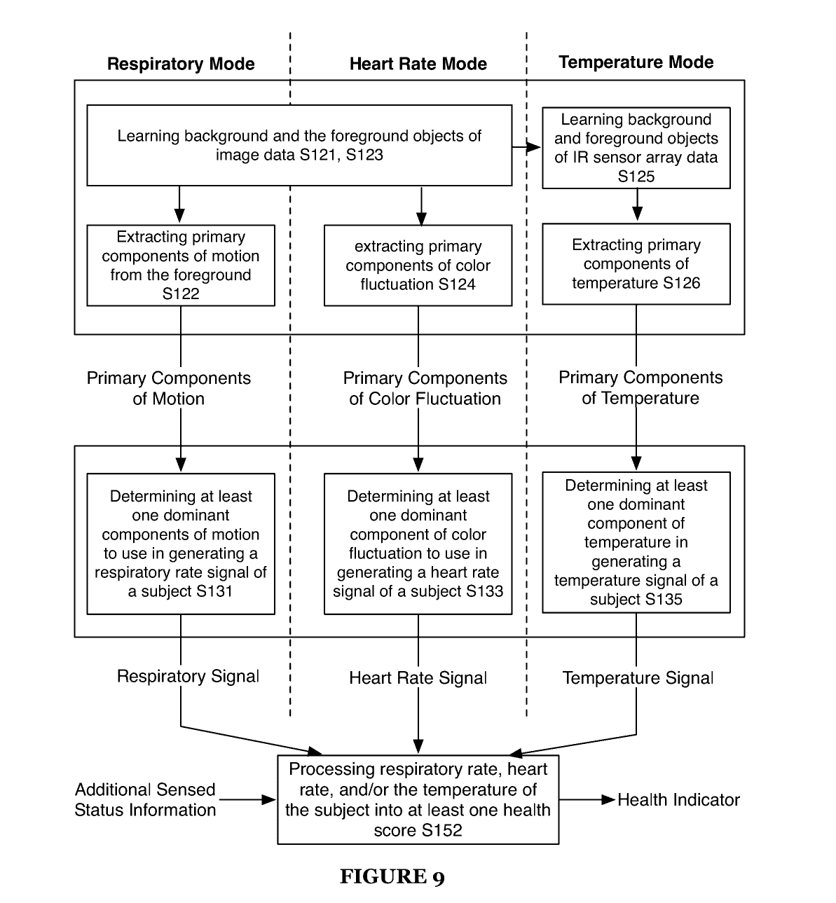

FIG. 9 is a detailed flowchart representation of sensing modes of a method of a preferred embodiment;

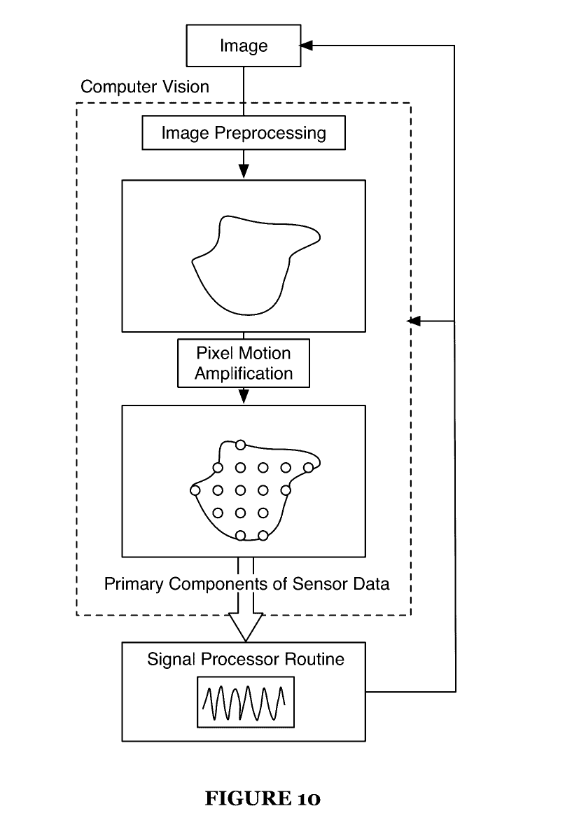

FIG. 10 is a detailed schematic representation of extracting and processing a set of primary components of motion;

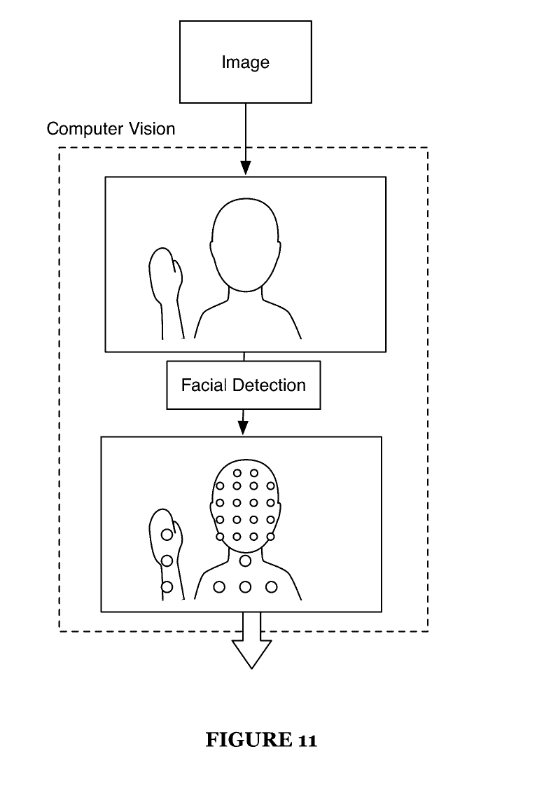

FIG. 11 is a schematic representation of prioritizing extraction of components of sensor data based on facial detection;

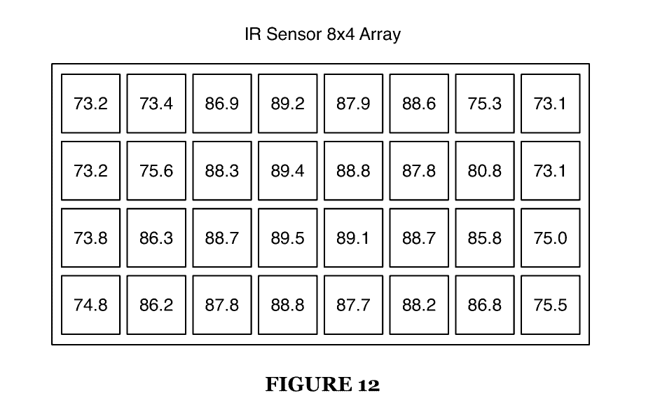

FIG. 12 is an exemplary set of IR sensor array data from an eight by four IR sensor array; and

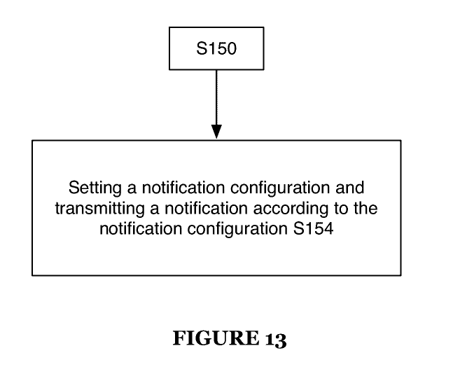

FIG. 13 is a flowchart representation of a variation a method with notifications.

### DESCRIPTION OF THE EMBODIMENTS

The following description of the embodiments of the invention is not intended to limit the invention to these embodiments but rather to enable a person skilled in the art to make and use this invention.

### 1. Overview

The system and method for monitoring biological status through contactless sensing functions to use remote sensing approaches to create an informative biological monitoring tool. The system and method is preferably entirely contactless using only remote sensing techniques without the use of an attachment to the subject. In other variations, the system and method may be used in addition to contact-based sensing such as in a hospital. In one embodiment, standard or customized video cameras may be used to enable biological status monitoring. In some variations, multiple biological sensing approaches can be used in collaboration to provide biological status monitoring powered by a set of biological traits of an observed subject or subjects. A multi-sensing variation of the system and method is preferably implemented through a multi-sensor video monitor system used in conjunction with a biological signal processing system. The system and method can be used in detecting respiratory rate, heart rate, and/or body temperature. Other suitable biological signals may be additionally or alternatively measured such as movement, sounds, and blood glucose levels. The biological signals can be used to determine a biological status, which can use multiple sources of information of a subject to provide an assessment of the person's (or animal's) state. The system and method can be used to perform multi-point sensing of one or more individuals. Additionally, the sensing techniques such as temperature can be applied to sensing and monitoring environmental conditions. Additionally, the remote, image-based sensing may be configured to monitor the biological status of multiple subjects simultaneously.

As a first objective, the system and method functions to provide remote biological monitoring of one or more subjects. The subjects may be passively observed without depending on the subject from performing some action or holding some position. For example, a n uncooperative baby could be a monitored subject.

As a second objective, the system and method can function to provide synchronized vital signs of one or more subjects through non-contact sensing. More specifically, the system and method can provide data on heart rate, respiratory rate, and/or body temperature. The system and method can additionally or alternatively provide data and sensing of multi-point temperature sensing (e.g., temperature of extremities), environmental temperature, motion detection, body position, awake-sleep detection, quality of sleep monitoring, visual monitoring, audio monitoring, and/or other forms of biological or environmental monitoring. The system and method is preferably implemented in association with at least one sensing device, which can be setup as a baby monitor camera/security camera.

As a third optional objective, the system and method can function to use multiple biological signals for cooperative feedback between at least two biological signals. As the contact-less sensing (i.e., remote sensing or near-proximity sensing) techniques may be susceptible to errors and/or inaccuracies in different scenarios, the system and method can use predictive modeling of one biological signal to alter the biological sensing and/or processing of a second biological signal. For example, a correlation between heart rate and breathing rate may be used to alter the predicted value of one rate based on the other rate.

As a fourth optional objective, the system and method can function to provide a health indicator for an individual. The system and method can transform the biological signal or signals of an individual to an easily interpretable indicator of general safety and health of an individual. The system and method can generate accurate and detailed measurements of biological information. In one variation, the biological data of the various signals is transformed into a single health indicator that accounts for multiple biological signals in a qualitative manner. In another variation, the biological data of each biological signal is transformed into a simplified health indicator. For example, an icon for each of an individuals heart rate, respiratory rate, and temperature can be colored icons that are green when within a healthy range, yellow when there may be an issue, and red when not in a healthy range. The health indicator may be based on current status but could additionally be based on biological data over a period of time. Additionally, the biological signals could be used in coordination with medical data parameters (e.g., recommended health parameters supplied by a pediatrician) to set automated notifications, alerts, or indicators when the biological signals go out of range.

As a fifth optional objective, the system and method can function to provide customized notifications of the well being of an individual. A user can customize thresholds for various notifications. The notifications can include in-app notifications, push notifications, phone calls, text/media messages, emails, automatic emergency calling, and/or any suitable form of notification. The notifications can be adaptive to different scenarios.

The system and method can be applied to a variety of different applications.

As a first exemplary application, the system and method can be applied to a baby/child-monitoring device. The system and method are preferably applied to a device monitoring a crib, bed, and/or resting area for baby or child. The system and method can be adjusted to provide assurances to a caretaker of a child's current state of well-being. Similarly, the system and method can be applied to monitoring pre-mature and critical condition infants. Such a system could similarly be used for monitoring of the elderly or sick. The system and method can alternatively be applied to monitor any suitable condition. For example, the system and method could be applied to sleep apnea. As another variation, the system and method can be used in monitoring non-human animals.

As a second exemplary application, the system and method can be applied to hospitalization. The system and method can be used to provide a nurse, doctor, or attendant a tool for remote monitoring of an individual patient. The system and method may alternatively be used in an implementation with a plurality of sensing devices. There could be a sensing device for multiple patients in different rooms. A patient-monitoring dashboard could allow an individual to simply monitor multiple patients. Notifications and alerts of different biological signal information can be brought to the attention of different hospital staff depending on the scenario. In another optional hospitalization scenario, the system and method could be used in triaging incoming patients by remotely monitoring biological signals as they enter the hospital or wait in the waiting room. Such hospitalization applications can similarly be applied to other use-cases. For example, the system and method could be used at prisons or institutions with at-risk individuals where remote sensing is preferred or even a requirement.

As a third exemplary application, the system and method can be used during field-diagnosis. Emergency crews, rescue robots, and/or other entities can use the sensing device to remotely detect biological signals of an individual in the field.

As a fourth exemplary application, the system and method can be used for biological surveillance. For example, the system and method can identify unhealthy passengers in an airport and possibly help in preventing the spread of disease. Additionally, a security application could detect a physiological presence. In a first variation a remotely sensed physiological presence may provide another aspect with which a security system could track and/or identify individuals. In another variation, the system and method could be used in evaluating the state of the physiological presence such as if the person is nervous, excited, scared, lying, relaxed, or any suitable meaningful information.

### 2. System for Monitoring Biological Status Through Contactless Sensing

As shown in FIGS. 1 and 2, a system for monitoring biological status through contactless sensing can include a sensing device interface 110 with a set of biological contactless sensors, a sensor data processing unit 210, a biological signal processor 220, and a monitor system 310. The set of biological contactless sensors preferably includes at least an interface with an video imaging source, but may additionally include an interface to an IR imaging source, radar inspection source, a 3D imaging source, and/or any suitable remote sensor source.

The sensing device interface 110 functions to provide a source for remotely sensed data that can be transformed into actionable biological statuses. In a first embodiment, the sensing device interface 110 functions as a communication interface with an outside or remote sensing device as shown in FIG. 1. The system may be implemented within processing service platform. Multiple entities could use the processing service platform to determine biological status of inspected subjects. A sensing device interface 110 with a remote sensing device may enable commodity or third party sensing devices to be used in combination with the system to provide biological status monitoring. The sensing device interface 110 can comprise a data stream of sensors of a sensing device. For example, a video camera already used with a surveillance system may integrated with the communication interface such that a video stream can be received through the sensing device interface 110 of the processing service platform and processed and transformed by the system. The sensing device interface 110 is preferably a network interface, which may utilize internet protocol based internet and/or intranet connections. Alternative wireless or wired communication connections may additionally or alternatively be used. For example, an institution such as a hospital or prison may use a network of cameras connected to an on-premise system operating on a centralized computing system.

In an alternative embodiment, part or all of the system is implemented in combination with a sensing device 120 included in the system, as shown in FIG. 2. The sensing device interface 110 of this embodiment may be internal to the sensing device or be a communication interface between the sensing device and a remote processing service platform. In some variations, a processing service platform may be used with customized sensing devices 120 of the system and with outside, third party sensing devices.

A sensing device 120 is preferably a wireless, network connected sensing device that includes an imaging device 130 and optionally an infrared (IR) sensor array 140. The sensing device 120 can include additional sensing components such as a microphone, ultra-wide band radar sensor, an ultrasound distance sensor, a structured light 3D scanner, 3D camera, and/or any suitable sensor. The sensing device 120 can collect data, detect vital signs, and identify physiological changes in a living organism. The sensing device 120 can interact with other components of the system to automate and integrate with various processes for vision perception and tasks enabling recognition of the living organism in question, motion estimation, and/or machine learning that allows for the extraction of meaningful biological assessment of a subject from a distance.

The sensing device 120 is preferably contained within a single housing as a single self-contained unit as shown in FIG. 3. In one implementation, the sensing device is a mountable sensing device that can be positioned to monitor a fixed area. The system can alternatively include a sensing device that is a network of multiple devices with duplicate or distinct sensing capabilities. For example, a temperature sensor can be in one unit and the heart rate sensor in a second unit. The sensing device 120 can additionally include a power supply, a processor, a communication/networking module, and/or any suitable components. Additionally components for a baby monitor variation may include a remote control or remote video monitor. The communication and networking module may provide an internet connection (e.g., e.g., Wi-Fi, Ethernet, etc.), cellular data connection, Bluetooth connection, a wired connection, and/or any suitable form of data connection. In one variation, part or all of the process routines of the sensor data processing unit 210 and/or biological signal processor 220 may be operable on the processor of the sensing device 120. For example, all sensor data transformation can be performed on the sensing device 120—the sensing device 120 could include a biological status output. In an alternative example, at least part of the sensor data processing unit 210 is operable on a processor of the sensing device 120—the processed data may be more efficiently streamed to a remote biological signal processor 220 for transformation to a biological signal.

The imaging device 120 functions to collect video of an individual. The imaging device 120 can collect video-based image data that can be applied to multiple sensing processes. The video data of the imaging device 120 can be directed to a respiratory computer vision process processor and a heart rate processor. The video data can additionally be used in object detection, facial detection/recognition, biomechanical modeling, or any suitable image processing technique. The imaging device can be a CCD, CMOS, or any suitable type of digital camera. In one variation, the imaging device can include an infrared camera mode for capturing images in low light or complete darkness. An infrared light emitting LEDs or other suitable light source (e.g., visible light LEDs) can be used to illuminate a subject such that respiratory rate and heart rate can be monitored.

The infrared (IR) sensor array 140 functions to perform contactless temperature sensing. The IR sensor array 140 can collect a matrix of temperature values. The data collected by the IR sensor array 140 is processed according to a temperature sensing process. In one variation, the internal temperature of the sensor, environmental temperature, temperature differential from the ambient temperature to the human temperature, and the distance between the IR sensor and the subject is factored into a generated subject temperature signal. The infrared sensor can be calibrated according to the distance from the subject. The distance may alternatively be automatically detected using a range finding sensors such as an ultrasound sensor, computer vision, or any suitable distance approximation technique. In one alternative, an individual IR sensor can be used in place of a sensor array.

The system may additionally or alternatively use a motion sensor. The motion sensor can preferably detect motion in substantially three dimensions. The motion sensor could be UWB radar, a structured light 3D scanner, a 3D imaging device, or any suitable sensing system that can be used in measuring or estimating motion. The motion sensor can be used in place of or in collaboration with the imaging device. For example, the respiration rate of a subject can be measured first using the video processing techniques applied to the 2D video data, and the respiratory rate of the subject can be measured through a second technique of detecting 3D movement in the chest region using UWB radar.

The sensing device can additionally include an audio microphone, the audio microphone could be used in detecting and analyzing environmental sounds. The audio microphone could alternatively be a directional microphone that can be targeted at an individual to detect minute audio signals. Amplified and isolated audio signals could be used in detecting respiration, heart rate, and/or other suitable biological functions.

The sensor data processing unit 210 functions to transform sensor data to a set of generalized analysis primitives from which biological signals can be generated by the biological signal processor 220. The system preferably uses the sensor data processing unit 210 cooperatively with the biological signal processor 220. The output of the sensor data processing unit 210 is communicated to the biological signal processor 220 to generate a biological signal. The sensor data processing unit 210 can include a set of sensor data extraction processes, wherein one or more sub-processes can be applied for different types of biological signals. The set of sensor data extraction processes can include a respiratory computer vision process 212, a temperature computer vision process 214, and/or a heart rate computer vision process 216. The sensor data processing unit 210 preferably converts at least part of the sensor data into a set of primary components of sensor data for each of the biological sensing modes used in the system. A component of sensor data is an extracted and processed set of sensor data that can be a primary candidate for possessing a detectable biological signal. Multiple sets of primary components of sensor data may be generated for each type of sensing mode.

The biological signal processor 220 functions to transform the data of the sensing device into one or more biological signals. There can be multiple stages of the processing. Preferably an initial stage is used to generate distinct biological signals, which can include a respiratory rate signal, a heart rate signal, a temperature signal, a blood glucose level signal, blood pressure signal, and/or any suitable biological signal. In the variation where the sensor data processing unit 210 extracts primary components of sensor data. The set of primary components of sensor data is processed to determine a dominant signal trend that relates to the biological signal. A biological signal will have general expected characteristics, which can be leveraged in identifying possible signals in the components of sensor data. In one variation, a first biological signal processing stage can use a second biological signal processing stage to augment the first biological signal processing stage. A final processing stage can create a health summary score, which can be a generalized assessment of the multiple biological signals.

In a first variation, the biological signal processor 220 can be located in a remote cloud hosted platform. The sensor device collects data and transmits the data to the cloud platform for processing. In a second variation, the biological signal processor system can be at least partially instantiated on the sensing device and at least partially instantiated in a remote host. The biological signal processor system may alternatively be instantiated in part or whole on any suitable device.

Similar to the sensor data processing unit 210, the biological signal processor 220 can include a set of biological signal processes, wherein one or more sub-processes can be applied for different modes of biological sensing of biological signals. The set of signal processes can include a respiratory biological signal process 222, a temperature biological signal process 224, and/or a heart rate biological signal process 226.

The respiratory computer vision process 212 is preferably used in combination with the respiratory biological signal process 222 when the system is used in a respiratory sensing mode. The temperature computer vision process 214 is preferably used in combination with the temperature biological signal process 224 when the system is in a temperature sensing mode. The heart rate computer vision process 216 is preferably used in combination with the heart rate biological signal process 228 when in a heart rate sensing mode. The various biological sensing modes can be enabled simultaneously such that different types of biological signals can be generated simultaneously. The respiratory related processes utilize components of motion from image data. The temperature related processes utilize components of temperature from an IR sensor array. The heart rate related processes utilize components of color fluctuation from image data.

The resulting biological signals may be used as outputs of the system, but may alternatively be processed collectively to generate a health indicator. Additional stages of processing within the system can additionally determine subject status information. In one variation status information is sleep status such as if a subject is currently awake, currently asleep, current stage of sleep, amount of sleep, and/or other properties relating to sleep. Status information could similarly be applied to activity, comfort level, coughing amount, and/or other aspects relating to the condition of a subject.

The monitor system 310 functions to inform an entity of the status of a monitored subject. The monitor system 310 can receive biological signal data and can additionally receive live or captured video, images, and/or audio from the sensing device. As shown in FIG. 4, in one variation, a live or captured video can be streamed along with abstracted health indicator representations of the biological signals. The monitor system 310 could include a standalone video and/or audio device used in outputting sensor information detected by the system. The monitor system 310 could alternatively be through an application or website accessible by a personal computing device such as a phone, computer, connected TV, a wearable device, or any suitable network-connected computer. In a first mode, the monitor system 310 shows biological signal data and video in substantially real-time such that an entity can actively monitor the subject. In a second mode, the monitor system 310 can provide alerts and/or notifications to events. The monitor system 310 can be configured to dynamically transmit notifications according to a health indicator. For example, instead of receiving periodic notifications with the biological signal, a user could receive notifications based on the priority status as determined by a health indicator. A notification could be a message transmitted over a communication channel but could alternatively be a visual indicator, audio indicator or any suitable alert for a user. The conditions triggering an alert or notification may be customized. For example, a parent may be notified when there is a change in the biological signals or environment of a sleeping baby. The monitor system 310 can additionally include user interface elements for receiving user input. The user input could be used in moving the sensing device, changing sensing properties, transmitting audio to a speaker connected to the sensing device, or performing any suitable action. For example, a parent or clinician could speak to a subject through network communicated audio played on a speaker of the sensing device.

In an alternative embodiment, a monitor dashboard may be used to monitor one or more sensing devices remotely over the internet. A monitor dashboard is preferably used to allow high-level monitoring of a set of sensing devices that are simultaneously monitoring multiple individuals.

### 3. Method for Monitoring Biological Status Through Contactless Sensing

As shown in FIG. 5, a method for monitoring biological status through contactless sensing can include collecting remote sensor data Silo, extracting primary components of sensor data for a set of biological sensing modes S120, processing the primary components of sensor data through a signal processing routine for a set of biological sensing modes to generate at least one biological signal S130, updating the extraction of primary components based on the signal processing routine S140, and applying at least one biological signal from the signal processing routine S150.

The method can be applied to various biological sensing modes, which may include detecting respiratory rate of a subject, detecting heart rate of a subject, and/or detecting temperature of the subject. The method can be used in generating one or more biological signals. The method may be used in reporting on biological status information related to respiratory rate, respiratory rate variability, respiratory patterns, movement patterns, heart rate, heart rate variability, heart rate patterns, temperature, temperature variability, using a combination to track aspects such as sleep status, sleep stage or comfort level, and/or any suitable biological status information. In one variation, a set of biological signals is used in combination to generate a comprehensive health score of a subject.

The method is preferably implemented by a multi-tenant processing service platform. The processing service platform can serve a plurality of accounts. Each account may have different types of sensor device architectures and/or utilize different features. For example, a first account may use a customized sensing device that includes customized sensing components and utilizes on-device processing, which may include extracting primary components of sensor data and transmitting the data characterization of the sensor data, and a second account may use a third party internet protocol type security camera (or any suitable imaging device). Multiple instances of the method can be performed simultaneously across multiple accounts.

The method may alternatively be performed by a system distributed between a sensing device and a processing service platform. Parts or all of method can be performed directly on a sensing device. Alternatively, a sensing device may stream information to a processing service platform. In one variation, the primary components of sensor data are extracted on a sensing device and then communicated to a remote cloud service. The media files may not need to be streamed. Alternatively, a local secondary device may process the primary components of sensor data before streaming to a remote cloud service. More specifically, the method can include communicating primary components of sensor data to the signal processing routine when extracting primary components of sensor data is performed at a sensing device and processing the primary components is performed at a remote computing device. The primary components of sensor data can be communicated wirelessly using Wi-Fi, Bluetooth, cellular data, or any suitable wireless communication channel. The primary components of sensor data could alternatively be communicated over a wired connection such as an IP-based wired connection.

Block S110, which includes collecting remote sensor data, functions to gather sensor data. The remote sensor data can include video imaging data collected from a camera. The remote sensor data may additionally or alternatively include IR sensor array data. Other forms of sensor data can include audio data, ultra-wide band radar sensor data, an ultrasound distance sensor data, structured light 3D scanner data, 3D camera data, and/or any suitable sensor data. In some variations, the sensor data is collected through a sensing device interface. The sensing device interface can be a wired or wireless connection. Preferably, the sensor data is streamed or communicated over a IP based network connection. In other variations, the method may be implemented in combination with one or more sensing devices actively collecting the sensor data.

Block S120, which includes extracting primary components of sensor data for a set of biological sensing modes, functions to reduce sensor data to relevant data samples that can be more readably processed. Performing remote sensing through a wide area, sensing device such as a video camera can result in collecting sensor data on all objects within the field of view of the camera. Many parts of the collected sensor data will have no relevance to the biological state of a subject. For example, when monitoring a baby, the video camera will preferably be directed at a crib and/or mattress where a baby may be sleeping, but it may additionally capture the floor, walls, other pieces of furniture, and any subject in the view. Additionally, the subject is not physically coupled to the sensing device and so extracting primary components of sensor data can be configured to compensate for varying orientation and movements of a subject. Extracting primary components of sensor data can involve manual selection of one or more regions and/or automated selection of one or more regions. Automated selection of regions can utilize object detection and/or learning background and foreground patterns within the sensor data.

For many biological sensing modes, one point of sensing (e.g., a single component of sensing data) can be used to determine a biological signal. This can include identifying a subject, determining a sample point on the subject, and conditioning sensor data of the sample point as shown in FIG. 6. In one variation, this can include performing face detection to identify a facial region of a subject, determining at least one sample point or a combination of pixel points in the facial region of the subject, and conditioning sensor data from the sample point on the facial region of the subject. Conditioning sensor data can include any suitable process, but generally includes a set of computer vision processing routines such as tracking pixel changes over time such as movement or color changes, amplifying pixel changes, and/or any suitable change. The resulting conditioning sensor data can act as a primary component of data and is communicated to a biological signal processor.

One sample point may be used to extract a primary component of sensor data. More preferably, multiple primary components of sensor data are extracted. In the face tracking implementation, multiple points may be sampled from the facial region, or multiple body features such as hands and arms can be automatically detected and used to determine a sample points. With multiple components of sensor data, the processing stage of block S130 can determine common themes across the components of sensor data, which may be more resilient in particular situations. If one sampling point became compromised (e.g., become blocked or less clearly offers remote sampling), the redundancy of sampling points can be used to maintain consistent biological signal monitoring. For each biological sensing mode, a different set of primary components of sensor data may be extracted. For example, respiratory sensing mode includes primary components of motion while heart rate sensing mode includes primary components of skin color changes due to blood perfusion in the skin.

Extracting primary components of sensor data can additionally include tracking objects within the sensor data. Tracking objects can compensate for slight or large movements of a subject. Primary components of sensor data can be updated to target different positions of image data based on the object tracking. Optical flow and/or other object tracking techniques can be used.

Block S130, which includes processing the primary components of sensor data through a signal processing routine for a set of biological sensing modes to generate at least one biological signal. Each sensing mode is preferably used to generate at least one biological signal. Some biological sensing modes may alternatively be used to generate multiple signals. For example, the temperature sensing mode can generate a body temperature signal and an environment temperature signal, and a heart rate sensing mode can generate a heart rate signal but can be augmented to additionally provide a blood oxygen saturation signal.

Processing the primary components of sensor data can include interviewing multiple primary components of sensor data and determining at least one dominant component of sensor data from which a biological signal is determined as shown in FIG. 7. In one variation, a biological signal may be generated for multiple components of sensor data and then the resulting biological signals can be compared when selecting a resulting biological signal. For example, of a set of components of sensor data, the biological signal with the greatest confidence level may be selected as the resulting output. In another variation, multiple components in combination may be used to generate the biological signal. For example a set of components can be combined into a composite component of sensor data.

Additionally processing the primary components of sensor data can include filtering the primary components of sensor data based on characteristics of the data. Filtering can be based on signal processing results of a component or any suitable factor. Filtering can utilize expected signal values, source disambiguation, eliminating noise, filtering based on historical variations, and/or analyzing signal intensity/power. Filtering primary components of sensor data filters out or otherwise deprioritizes components with characteristics outside of biological signal expectations. Primary components of sensor data and/or biological signals from a primary component of sensor data that do not fit within biological signal expectations filtered out and are preferably not considered. For example, a heart rate is expected to be within a certain threshold and to have some maximum rate of change—components that result in signals outside of this may be ignored and deprioritized.

Extracting and processing primary components of sensor data can additionally promote monitoring of multiple subjects as shown in FIG. 8.

The set of biological sensing modes can include one or more sensing modes. Three preferred sensing modes that can be implemented individually or as a collection include a respiratory rate mode, a heart rate mode, and a temperature mode.

As shown in FIG. 9, for a respiratory rate sensing mode, the method can be applied to detecting the respiratory rate of a subject, wherein the method comprises learning background and the foreground objects of image data S121, extracting primary components of motion from the foreground S122, and determining at least one dominant components of motion to use in generating a respiratory rate signal of a subject S131. Detecting respiratory rate of a subject, functions to detect breathing motions of a subject and calculate a breathing rate. Detection of respiratory rate can be achieved by monitoring a video sample from the chest region of a subject. In some cases, however, identifying the chest region can be challenging. The extraction of primary components of motion can account for the complexities of identifying a sampling point. The sensor data is preferably image data, which may include color visible light image data, IR image data, 3D image data, and/or any suitable type of image data. Image data can be prepared or processed in a variety of approaches prior to subsequent computer vision processing. In one implementation, mage data could be converted to grayscale and equalized across a histogram.

Foreground and background learning preferably utilizes historical data but may additionally use heuristical or user input approaches to segmenting background and foreground objects within the image data. For example, in the baby monitor use-case scenario, the static objects in the view such as the crib and walls will be learned and classified as background, while the baby and possibly newly introduced objects such as a blanket or toy may be extracted as foreground objects. Various computer vision techniques such as blob detection may be applied to determine foreground objects. Various regions or points of the foreground object are then used as primary components of motion.

A primary component of motion is preferably a resulting data characterization of the movement properties for a particular region. For the primary components of motion that can be suitably used to generate a respiratory rate signal, there will be slight pixel movements induced by respiratory activity of a subject.

The primary components of motion are preferably extracted from the foreground objects. A primary component of motion is preferably associated with a point, region, or object with a location in the image data. Various regions, points, or locations across the foreground object may be referenced when generating the primary components of motion. In one variation, a regular sampling of regions is used. In another variation, object detection and/or subject modeling may be applied to prioritize or otherwise augment the determination of a set of primary components of motion. In one variation, the sample region of a foreground object may be determined by identifying a chest region of a subject. The sample area can be identified through modeling of the subject. In one variation, facial detection is used to identify the head as a reference object, and then modeling out where the chest is located. Alternative approaches to identifying a sample area may be used. In general, a flexible open sampling of foreground objects can be used, while the determination of the utility of the components is delegated to the signal processing stage of block S130. Open and regular sampling of foreground objects may automatically enable simultaneous biological signal monitoring of multiple subjects within the field of view of the image data.

In one variation, pixel motion amplification can be applied. Amplifying pixel motion is preferably performed prior to extracting the primary components of motion as shown in FIG. 10.

Additionally, the method can include tracking motion and augmenting the primary components of motion to compensate. Augmenting the primary components of motion preferably includes adjusting the associated positioning of the primary component of motion. In one variation, optical flow can be used. The primary components of motion are preferably repositioned to map to the same point on the subject. For example, a primary component of motion associated with the right shoulder can be repositioned as the subject rolls over. Motion of a subject can be isolated by identifying the number of pixels showing significant movement in the sample area. In one variation, the amount of amplification can be customized depending on the environment.

Determining at least one dominant component of motion to generate a respiratory rate includes analyzing the set of components of motion to determine the strongest signal that corresponds to a reasonable respiratory signal. Filtering of results can be used to eliminate signals out of a reasonable range. Segmenting may additionally be used. In an ideal scenario the respiratory rate signal is corroborated across multiple primary components of motion. For example, movement caused by respiratory activity on the chest will likely be similar to movement on the stomach. In some cases multiple strong signals may be determined, which can be a result of multiple subjects being within view.

As shown in FIG. 9, for a heart rate sensing mode, the method can be applied to detecting heart rate of the subject, which includes learning background and foreground objects of image data S123, extracting primary components of color fluctuation S124, and determining at least one dominant component of color fluctuation to use in generating a heart rate signal of a subject S133. Detecting heart rate of the subject functions to detect changes in the skin properties of a subject to extract a heart rate. Additional, aspects such as blood oxygen saturation levels may additionally or alternatively be extracting using the analysis of skin color changes caused by blood activity. The detection of a heart rate preferably uses a process of photoplethysmology using video from an imaging device. Similar to the respiratory sensing mode, the sensor data is preferably image data, which may be preprocessed in similar or alternative approaches. While respiratory activity may be sensed through clothes, heart rate sensing preferably relies on viewing of color changes present in skin.

Extracting primary components of color fluctuation functions to generate primary components of skin color viewed over time. Uniform, open sampling of foreground objects may similarly be used for the primary components of color fluctuation. Regions of no interest may be included within the primary components of color, but such components of color fluctuations will preferably be filtered or deprioritized during the biological signal processing step. Additionally or alternatively subject body part detection and/or modeling may be used to prioritize or select regions for the primary components of color fluctuation. In one variation, a region of a primary component of color fluctuation can be identified by detecting a face and selecting or prioritizing an exposed area of skin based on the detected face as shown in FIG. 11. Detecting a face can include a Haar Cascade facial detection approach but any suitable facial detection approach may be used. Selecting an exposed area of skin preferably includes selecting an area of the subject's forehead.

A primary component of color fluctuation is preferably a resulting data characterization of color at a particular points. A useful component of color fluctuation will be associated with a region of skin of a subject. More specifically, the data characterization is an extracted spectrum channel. Extracting a spectrum channel can include extracting any suitable color channel of the image set such as extracting the green channel or the red channel from an RGB color space. In a variation where the image data is in a YIQ color space, the intensity channel may be used. A selected spectrum of the image may additionally be magnified. The magnification of a selective spectrum of the image can be based on optical flow estimates to determine the variability of color and amplifying the color based on the regions identified by optical flow detection. Magnification can consist of shifting the range of the spectra to either extremes to differentiate the variability of color. The amount of shifting can be dependent on environmental conditions or the image composition including skin tone.

Determining at least one dominant component of color fluctuations includes analyzing the set of components of color fluctuation to determine the strongest signal that corresponds to a reasonable heart rate signal. Filtering of results can be used to eliminate signals out of a reasonable range. Segmenting may additionally be used. In an ideal scenario the heart rate signal is corroborated across multiple primary components of color fluctuations. For example, the skin color changes on the forehead will likely be similar to skin color changes on the temple. In some cases multiple strong signals may be determined, which can be a result of multiple subjects being within view.

As shown in FIG. 9, for a temperature sensing mode, the method can be applied to detecting temperature of the subject, which in one implementation includes learning background and foreground objects of IR sensor array data S125, extracting primary components of temperature S126, and determining at least one dominant component of temperature in generating a temperature signal of a subject S135. Detecting temperature of a subject functions to use IR sensing to remotely measure the body temperature of an individual. Detecting temperature of the subject preferably includes collecting IR light data from an IR sensor array that is targeted at the subject as shown in FIG. 12. The IR sensor array preferably collects a sample of IR light data from a skin area of the subject. In another variation, the IR sensor array collects a set of IR light data from distinct areas of a subject's skin region. The set of IR light data can be used to create a set of temperature data samples as multiple components of temperature.

Learning background and foreground IR sensor array data functions to segment the IR sensor data between background objects and possible subjects. In one variation, The IR sensor array data can be used in combination with image data. The IR sensor data is preferably an image map of a matrix of IR sensing points collected from an IR sensor array. The IR sensor array data and the image data preferably have sufficiently overlapping fields of view such that one point of the IR sensor data maps to a point or region of the image data. The IR sensor data may have less resolution than corresponding image data. Background and foreground learning in blocks S121 or S123 may be initially performed on image data as described above and then mapped to the IR sensor data.

Additionally or alternatively, temperature difference analysis across the IR sensor array data can be used to extract primary components of temperature. For example, a person can be automatically identified by the IR sensor array by observing the temperature differential among the infrared sensors. Additionally or alternatively, face detection and subject modeling can be used to select or prioritize particular locations for use as primary components of temperature.

In addition to using background and foreground segmentation, a subset of IR sensor array data may be deprioritized or even disqualified. In some cases, pixels of the IR sensor array provide unreliable data, which may be determined over time or through calibration. The unreliable pixels are preferably not considered.

A primary component of temperature is preferably a characterization of IR sensor data as a temperature measurement over time. Determining a dominant component of temperature can function in a similar manner to determining a dominant component for respiratory or heart rate except wherein the primary components of temperature are processed for temperature analysis.

Additional parameters may be used in generating the primary components of temperature, determining a dominant component of temperature, or generating a temperature signal. Additional parameters could include internal temperature of the sensor, environmental temperature, temperature differential from the ambient temperature to the human temperature, median temperature range, and/or distance between the sensor and the human subject. This information can be correlated to the information from the camera to estimate the temperature in different regions of the body and the temperature variations can be used to estimate vitals. For example, the variations in temperature in the nasal region can be measured and correlated with respiration. Additionally, the detection of temperature can be used in detecting temperature of environmental elements such as the room or an object.

Block S140, which includes updating the extraction of primary components based on the signal processing routine as shown in FIG. 10, functions to use the biological signal processing stage to augment the manner in which a primary component of sensor data are extracted. In one variation, a result of a signal processing routine is used to alter preprocessing of sensor data, the computer vision processing routine, the classification as background and foreground objects, the location of a primary component of sensor data, and/or any suitable factor. In one implementation, the result of a signal processing routine is used to alter the collection of sensor data. For example, the sample rate, sensing properties, and/or any suitable property may be altered.

The method can additionally include building patient models. A patient model is preferably a characterization of biological patterns in one or more biological signals. A patient model can be built specifically for an individual. In one variation, Haar based cascade classifiers can be used in building a model of different configurations of a patient object. A patient model may alternatively be built for a type of patient such as an infant, a child, a senior citizen. A patient model can additionally be built for specific classes of individuals such as patients suffering from particular ailments.

Additionally, the patient model could be adaptive and be automatically updated based on the health or biological progress of an individual. For example, the method could include acquiring the biological status of a newborn. Acquiring the biological status can include collecting weight, height, and age. The patient model of this individual could be automatically updated to correspond to the age, weight, and/or height of the patient. Information such as weight and/or height may be estimated through the method but can additionally or alternatively be set manually by the user at periodic increments.

The method can additionally include extracting a biomechanical model of an individual. The biomechanical model can be applied to identifying where in the image data to sample for the biological samples. For example, temperature and/or heart rate are preferably detected by sampling an image area of the skin of an individual. Facial detection can be a basic approach to determining where to sample, but the biomechanical model may be used as a supplemental approach or as a replacement approach, such as in situations where a face is not visible. Haar based cascade classifiers or using convolutional neural networks are exemplary variations of training images. Respiratory sampling may additionally want to sample areas of the chest where breathing motion would be most prevalent. The biomechanical model could additionally provide other information that can be used in monitoring a subject. For particular subjects, it may be desired to have the subject sleep or rest in a particular position. For example, a newborn preferably sleeps on her back. The biomechanical could detect if a baby is sleeping in an undesired position. Additionally, the biomechanical model could be used in understanding the interaction of the subject to environmental elements. For example, applying the biomechanical model in addition to other computer vision techniques, the method could identify if an object is obstructing a subject's breathing such as if a baby pulls a blanket over her face or rolls face down.

The method can additionally include detecting particular actions such as shivering, rolling, crying, chewing, waking up (e.g., eyes open), falling asleep (e.g., eyes closing), being content (e.g., calm facial expression and/or smiling), being anxious or upset (e.g., frowning or being agitated).

Block S150, which includes applying at least one biological signal from the signal processing routine, functions to output at least one resulting biological signal for a present subject. Multiple biological signals of different types may generated for a single subject. Alternatively, biological signals of the same type may be generated for multiple subjects. The respiratory sensing mode, the heart rate sensing mode, the temperature sensing mode, and/or subject state sensing mode may be used in any suitable combination. Within a multi-tenant processing service platform, different accounts may have different combinations of sensing modes enabled.

A biological signal can be applied in a variety of ways such as generating a comprehensive health score, generating notifications or alerts, providing access, generating recommendations for behavior training (e.g., changes to sleep), predictive analytics on biological status changes, long term training of normal biological patterns, and/or any suitable application.

In one variation, applying the at least one biological signal S150 includes processing respiratory rate, heart rate, and/or the temperature of the subject into at least one health score S152 as shown in FIG. 9, which functions to use the detected signals of the subject and environment to understand the status of the subject. In one variation, a generic health score can be calculated based on the current and past biological signals. A single health score could be used as a simple metric that can be used to determine when and if an individual monitoring the subject should check on the subject. In the case of a baby monitor, the health score can be a metric used to communicate to a parent when to check on the baby. A general health score can simplify the sensed data from a set of quantitative measurements into a qualitative status indicator. The general health score indicator can be a user feedback element to communicate the general health score. In one exemplary implementation, the feedback element can be a graphical indicator wherein the color indicates the health status with three states: normal, warning (e.g., check on the baby), and emergency (e.g., possible health risk). For example, a small icon could be displayed on a wearable device such as a smart watch where the parent can verify that their child is safe with only a glance. The user feedback element can be an audio user feedback element, a status message, or any suitable form of user feedback.

In another variation, a set of health scores can be generated. In one variation, an individual health score can be generated for each of the respiration rate, heart rate, and temperature signals. As with the general health score these individual health scores can transformed into a qualitative indicator. For example, three icons for each health score can be shown. Color, pulsing animations, audio, or other user feedback elements can be used to indicate their state. In one variation, the health score can be used to generate recommendations. The recommendations may be directed towards patient or care giver behavior changes. In one particular application, the health score may be generated as a reflection of sleep patterns. The generated recommendations could be recommended changes in sleep patterns such recommendations related to sleep position, bed time, mattress, sound proofing, sleep apnea devices, room temperature, and/or other recommendations.

The set of biological and/or environmental elements measured by the sensing device can be used collectively to augment the individual calculation of a given signal. The collective processing of the different signals can use the correlation between different biological processes to be reflected in one or more health scores. Collective processing additionally can accommodate possible inaccuracies of the contactless sensing techniques. In certain situations, one or more of the sensing techniques may provide inaccurate information. This may be detected and accounted for in the processing and/or collection of other biological sensing processes. For example, if respiratory rate appears normal but heart rate is low, the sensing of the heart rate can be updated as to where the data is sampled on the subject.

In another variation, applying the at least one biological signal S150 includes setting a notification configuration and transmitting a notification according to the notification configuration S154 as shown in FIG. 13, functions to enable customized alerts. The method can use the combination of multiple biological signals and environmental aspects to intelligently alert one or more individuals of the condition of a monitored subject. A default set of notifications and alerts can be configured for a sensing device. However, a user may be able to set customized notification rules. The user may add what notifications are used and/or the thresholds at which they trigger. The notification configurations can be based on one or more health scores but can additionally be based on the underlying biological signal data. The notifications can additionally or alternatively be dynamic according to the state of the subject. In a first variation, the notification configurations of a baby dynamically change with the age of the baby. For example, the thresholds and types of notifications can be different during the first week of life and the second month.

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

We claim:

1. A system comprising:
a sensing device comprising at least one video imaging device;
a sensor data processing unit, wherein the sensor processing unit when in a respiratory sensing mode is configured to extract a set of primary components of motion from image data from the video imaging device;
a biological signal processor that when in a respiratory sensing mode is configured to identify at least one dominant component of motion and generate a respiratory signal; and
a monitor system.

2. The system of claim 1, wherein the sensing device further comprises an infrared sensor array; wherein the sensor data processing unit when in a temperature sensing mode is configured to extract a set of primary components of temperature from data of the infrared sensor array; wherein the biological signal processor when in a temperature sensing mode is configured generate a temperature signal; and wherein the temperature sensing mode and the respiratory mode are enabled simultaneously.

3. The system of claim 2, wherein the sensor data processing unit when in a heart rate sensing mode is configured to extract a set of primary components of color fluctuations from image data from the video imaging device; wherein the biological signal processor when in a heart rate sensing mode is configured to identify at least one dominant component of color fluctuation and generate a heart rate signal; and wherein the temperature sensing mode, the respiratory mode, and the heart rate sensing mode are enabled simultaneously.

4. The system of Claim 3, wherein the biological signal processor is configured to process the respiratory signal, the temperature signal, and the heart rate signal to generate at least one health indicator.

5. The system of Claim 4, wherein the monitor system is configured to automatically transmit notifications according to the at least one health indicator.

6. The system of Claim 5, wherein the sensing device additionally includes a microphone; and wherein the biological signal processor additionally generates a subject status signal that at least partially relates to sleep status of the subject.

7. The system of claim 1, wherein the biological signal processor is configured to identify multiple distinct dominant components of motion and generate a respiratory signal for each dominant component of motion.

8. The system of claim 1, wherein the sensor data processing unit is physically mounted within a housing of the sensing device; wherein the biological signal processor is instantiated on a remote cloud computing system; and wherein at least the primary components of motion are communicated from the sensing device to the biological signal processor.

9. A processing service platform operable with a set of accounts, wherein for at least one account the processing service platform comprises:
a sensing device interface that receives at least video image data of an account;
a sensor data processing unit, wherein the sensor processing unit when in a respiratory sensing mode is configured to extract a set of primary components of motion from the video image data;
a biological signal processor that when in a respiratory sensing mode is configured to identify at least one dominant component of motion and generate a respiratory signal; and
a monitor system.

10. The processing service platform of Claim 9, wherein the sensing device interface additionally infrared sensor array data; wherein the sensor data processing unit when in a temperature sensing mode is configured to extract a set of primary components of temperature from infrared sensor array data; wherein the biological signal processor when in a temperature sensing mode is configured generate a temperature signal; and wherein the temperature sensing mode and the respiratory mode are enabled simultaneously.

11. The processing service platform of claim 10, wherein the sensor data processing unit when in a heart rate sensing mode is configured to extract a set of primary components of color fluctuations from the video image data; wherein the biological signal processor when in a heart rate sensing mode is configured to identify at least one dominant component of color fluctuation and generate a heart rate signal; and
wherein the temperature sensing mode, the respiratory mode, and the heart rate sensing mode are enabled simultaneously.

12. The processing service platform of Claim ii, wherein the biological signal processor is configured to process the respiratory signal, the temperature signal, and the heart rate signal to generate at least one health indicator.

13. The processing service platform of Claim 9, wherein the biological signal processor is configured to identify multiple distinct dominant components of motion and generate a respiratory signal for each dominant component of motion.

14. A method for contactless biological monitoring comprising:
collecting sensor data that comprises collecting imaging data of at least one subject;
extracting primary components of sensor data by a computer vision process;
processing the primary components of sensor data at a signal processing routine;
wherein extracting primary components of sensor data comprises extracting a set of primary components of motion through a computer vision process for a respiratory sensing mode;
wherein for a respiratory sensing mode, processing the primary components of sensor data comprises processing the primary components of motion to determine a respiratory signal for at least one subject; and
updating at least one part of extracting primary components of sensor according to the processing of the primary components of sensor data.

15. The method of claim 14, wherein extracting a set of primary components of motion comprises learning background and foreground objects of the image data, amplifying pixel motion of foreground objects, and extracting primary components of motion from the foreground.

16. The method of claim 14, wherein collecting sensor data further comprises collecting infrared sensor array data; wherein extracting primary components of sensor data when in a temperature sensing mode comprises extracting a set of primary components of temperature from the infrared sensor array data; and wherein processing the primary components of sensor data when in a temperature sensing mode comprises processing the primary components of temperature for at least one subject.

17. The method of claim 16, wherein extracting primary components of sensor data when in a heart rate sensing mode comprises extracting a set of primary components of color fluctuation from the infrared sensor array data; and wherein processing the primary components of sensor data when in a temperature sensing mode comprises processing the primary components of color fluctuation to determine a dominant component of color fluctuation and generating at least one heart rate signal.

18. The method of claim 14, wherein processing the primary components of sensor data comprises filtering out components of sensor data with characteristics that are outside of biological signal expectations.

19. The method of claim 14, further comprising wirelessly communicating primary components of sensor data to the signal processing routine; wherein extracting primary components of sensor data is performed at a sensing device and processing the primary components is performed at a remote computing device.
